(19) United States
(12) Patent Application Publication
(10) Pub. No.: US 2010/0054988 A1
(43) Pub. Date: Mar. 4, 2010

(54) PHOTOCATALYTIC NANOCAPSULE AND FIBER FOR WATER TREATMENT

(76) Inventor: Kwangyeol Lee, Namyangju-si (KR)

Correspondence Address: KNOBBE MARTENS OLSON & BEAR LLP, 2040 MAIN STREET, FOURTEENTH FLOOR, IRVINE, CA, 92614, US

(21) Appl. No.: 12/202,059

(22) Filed: Aug. 29, 2008

(51) Int. Cl. B01D 39/00; B01J 21/08; B01D 53/86; C02F 1/72

(52) U.S. Cl. 422/4; 502/232; 428/378; 428/311.11; 210/763; 977/773

(57) ABSTRACT

Systems and methods of forming photocatalytic nanocapsules and photocatalytic fibers are disclosed. The methods can include encapsulating one or more photocatalytic nanoparticles in a shell including at least one nanopore. The methods can further include forming photocatalytic fibers from a solution having one or more photocatalytic particles in a polycarbosilane melt.

### BACKGROUND

Pollution of aqueous solutions and air is an ever expanding problem in the modern world. An ever-growing number of toxic pollutants are produced by industries, such as, for example, textile industries, chemical industries, pharmaceutical industries, pulp and paper industries, and food processing plants. The majority of these toxic pollutants are released within two primary fluid physical states: water and air. As the scope of water and air-borne pollutant production increases worldwide, the dangers imposed by these released pollutants on the environment also increases. Additionally, environmental regulations are requiring that these released fluid streams contain less and less pollutants. In fact, some treatment processes that were acceptable options at one point in time are now obsolete because lower treatment standards are required as new environmental regulations are implemented on the state and federal level.

A variety of wastewater purification methods have been developed. Some techniques for removing the contaminants involve use of strong oxidants, which may themselves be hazardous. Other techniques remove the contaminant from the fluid but then release the contaminant into the air or produce a contaminant output, which must be disposed of.

### SUMMARY

In some aspects, there can be photocatalytic nanocapsules that can include one or more photocatalytic nanoparticles. The photocatalytic nanocapsules can further include a shell at least partially composed of silicon dioxide. The shell can at least partially encapsulate the one or more photocatalytic nanoparticles and can include at least one nanopore. The one or more photocatalytic nanoparticles can include, for example, one or more titanium dioxide nanoparticles. The one or more photocatalytic nanoparticles can include, for example, one or more of ZnO, CdS, SrTiO₃, Fe₂O₃, V₂O₅, SnO₂, FeTiO₃, PbO, other photocatalytic materials, and combinations of the same. The one or more photocatalytic nanoparticles can be one or more doped photocatalytic nanoparticles. The shell can be configured to allow organic matter to enter through the shell through the at least one nanopore. The shell can be hydrophilic, for example.

In other aspects, there can be methods of sterilizing or cleaning, which methods can include, for example, contacting a substance that includes organic matter with a plurality of photocatalytic nanocapsules described above. The photocatalytic nanocapsules can decompose the organic matter. The substance can include water. The methods can further include exposing the plurality of photocatalytic nanocapsules to light. The light can include visible light, for example.

In other aspects, there can be photocatalytic fibers that can include one or more photocatalytic nanoparticles. The photocatalytic fibers can further include a fiber that includes silica, for example. The silica can at least partially surround the one or more photocatalytic nanoparticles. The photocatalytic nanoparticles can include or be in the form of one or more nanorods. The photocatalytic nanoparticles can include titanium dioxide, for example. The nanoparticles can include one or more of ZnO, CdS, SrTiO₃, Fe₂O₃, V₂O₅, SnO₂, FeTiO₃, PbO, other photocatalytic materials, and combinations of the same. The nanoparticles can be organic. The nanoparticles can be water soluble. The photocatalytic nanoparticles can include a surfactant. The surfactant can at least partially coat the nanoparticles. The fiber can be a nanofiber.

In other aspects, there can be a plurality of fibers that can include at least one photocatalytic fiber described above. The plurality of fibers can further include at least one non-photocatalytic fiber. The non-photocatalytic fiber can include cotton, for example. The photocatalytic fiber can include a titanium dioxide nanoparticle, for example. The photocatalytic fiber can include one or more of ZnO, CdS, SrTiO₃, Fe₂O₃, V₂O₅, SnO₂, FeTiO₃, PbO, other photocatalytic materials, and combinations of the same. In other aspects, there can be filters that include the plurality of fibers described above and elsewhere herein. The plurality of fibers can be or include a bundle of fibers.

In other aspects, there can be methods of filtering that can include contacting a filter that includes one or more photocatalytic fibers that include one or more photocatalytic nanoparticles with fluid for filtration. The fluid can traverse or pass through the filter and contact the one or more photocatalytic nanoparticles. Contaminant particles in the fluid can be oxidized by one or more photocatalytic nanoparticles. The fluid can include water, air and/or any other liquids or gases, for example.

In other aspects, there can be methods of making a photocatalytic nanocapsule. The methods can include forming a photocatalytic nanocapsule, for example, using an emulsion technique. The photocatalytic nanocapsule can have one or more photocatalytic nanoparticles at least partially surrounded by a silicon dioxide shell. The methods can further include purging the photocatalytic nanocapsule with air to remove organic content. The methods can further include etching in a basic buffer solution to enlarge one or more nanopores of the photocatalytic nanocapsules. The methods can further include forming the photocatalytic nanoparticles by hydrolyzing alkoxide in solution. The photocatalytic nanoparticles can be formed in the presence of a non-photocatalytic metal oxide or metal sulfide. The photocatalytic nanoparticles can include one or more titanium oxide nanoparticles. The methods can further include forming a plurality of photocatalytic nanocapsules using an emulsion technique, where each photocatalytic nanocapsule can include one or more photocatalytic nanoparticles and a silicon dioxide shell surrounding the photocatalytic nanoparticles.

In other aspects, there can be methods of forming a photocatalytic fiber. The methods can include, for example, dispersing one or more photocatalytic nanoparticles in a polycarbosilane melt. The methods can further include forming at least one photocatalytic fiber from a solution that includes photocatalytic nanoparticles in the polycarbosilane melt. The formation of the photocatalytic fiber from the solution can include a melt spinning process. The fiber can be a nanofiber, for example. The nanoparticle can be in the form of a nanorod, for example. The photocatalytic nanoparticle can be at least partially composed of titanium dioxide nanoparticles. The photocatalytic nanoparticle can be at least partially composed of one or more of ZnO, CdS, SrTiO₃, Fe₂O₃, V₂O₅, SnO₂, FeTiO₃, PbO, other photocatalytic materials, and combinations of the same. The photocatalytic nanoparticle can be organic. The photocatalytic nanoparticle can be water soluble. The nanoparticle can include a surfactant coating.

In other aspects, there can be methods of forming a photocatalytic filter. The methods can include, for example, dispersing one or more photocatalytic nanoparticles in a polycarbosilane melt. The methods can further include forming one or more photocatalytic fibers from a solution including the one or more photocatalytic nanoparticles in the polycarbosilane melt. The methods further can include bundling the one or more photocatalytic fibers with one or more non-photocatalytic fibers. The photocatalytic nanoparticles can include one or more titanium dioxide nanoparticles. The photocatalytic nanoparticles can include one or more of ZnO, CdS, SrTiO₃, Fe₂O₃, V₂O₅, SnO₂, FeTiO₃, PbO, other photocatalytic materials, and combinations of the same. The non-photocatalytic fibers can include cotton. The bundling can include interweaving or intertwining the fibers, for example.

The foregoing is a summary and thus contains, by necessity, simplifications, generalization, and omissions of detail; consequently, those skilled in the art will appreciate that the summary is illustrative only and is not intended to be in any way limiting. Other aspects, features, and advantages of the devices and/or processes and/or other subject matter described herein will become apparent in the teachings set forth herein. The summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIGS. 1A-1D show cross-sectional views of various illustrative photocatalytic nanocapsules that contain photocatalytic nanoparticles for at least partially degrading a contaminant.

FIG. 2A shows an illustrative photocatalytic nanocapsule in which one contaminant can fit inside the nanocapsule.

FIG. 2B shows an illustrative photocatalytic nanocapsule in which two contaminants can fit inside the nanocapsule.

FIG. 3 shows an illustrative process of making a photocatalytic nanocapsule.

FIG. 4 shows an illustrative photocatalytic fiber that includes one or more photocatalytic particles.

FIG. 5 shows an illustrative process of making a purification system, e.g., filter, that includes photocatalytic fibers intertwined with non-photocatalytic fibers.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Aspects of the present disclosure relate, inter alia, to catalytic materials, including photocatalytic nanomaterials and systems, as well as to methods of making and using the same. The photocatalytic materials and systems can include photocatalytic nanoparticles. In some aspects the materials, systems and methods can provide more efficient and economic fluid purification methods. In some aspects the materials, systems and methods relate to effective and convenient filtering of contaminants from fluids, for example. The word contaminant can broadly include any physical, chemical, biological, or radiological substance or matter that has an adverse effect on air, water, or soil, including substances that would make the air, water or soil unfit for certain uses or for consumption, as well as substances that are otherwise desired to be avoided (e.g., chemical or biological waste). Contaminant can also refer to other substances that are simply not desired or that are desired to be removed for any reason, even though the substances may not necessarily be have an adverse effect on air, water, soil, etc.

In some aspects instance, at least one photocatalytic nanoparticle can be at least partially surrounded, for example, by a shell (e.g., silicon dioxide). A contaminant may enter through the shell's exterior and become “trapped” within the shell. The contaminant may become trapped, for example, in a pore within the shell. The nanoparticle may then contact the contaminants and may act, for example, as a photocatalyst to degrade or decompose the contaminant. In some embodiments, a fiber can include one or more photocatalytic nanoparticles and an adsorbent (e.g., silica) that at least partially surrounds the photocatalytic nanoparticles. Thus, fluid molecules may collect on or contact the fiber and contact the photocatalytic nanoparticles. The photocatalytic nanoparticles may then degrade the contaminant, e.g., based on the photocatalytic activity described above.

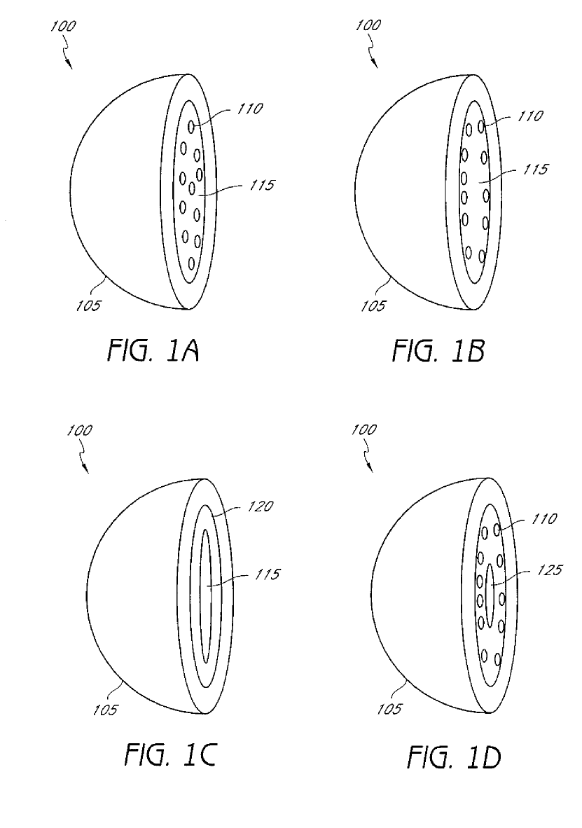

FIGS. 1A-1D show cross-sectional views of various illustrative photocatalytic nanocapsules that include one or more photocatalytic nanoparticles for at least partially degrading or decomposing a contaminant. The nanocapsule 100 may include a shell 105, one or more nanoparticles 110, cavity 115, photocatalytic nanoparticle layer 120 (FIG. 1C) and contaminant 125. The shell 105 may be semi-permeable or permeable (e.g., to a fluid, liquid, gas, air, water, specific contaminants, and/or organic matter). The shell 105 may be non-porous, semi-porous or porous. Methods of making porous shells are known in the art. See, for example, Accounts of Chemical Research Vol. 35, No. 11, 2002, which is incorporated herein by reference in its entirety. In some embodiments, the shell can include substantially no holes or openings, in that the shell forms a complete enclosure. In other embodiments, the shell does include at least one hole or opening, and optionally contains multiple openings. The shell 105 may include one or more polycrystalline materials. The shell may be adsorbent or at least partially adsorbent. The shell 105 may include a metal oxide and/or silicon (e.g., silicon dioxide). The materials in some aspects can permit light to contact photocatalytic nanoparticles within the nanocapsule 100. The shell may be hydrophilic, amphiphilic or hydrophobic. The shell may be in any suitable shape, such as but not limited to a spherical or ellipsoidal shape, for example. Other shapes are contemplated, including but not limited to irregular shapes or shapes that are partially or imperfectly shaped or geometrically shaped.

The shell may have a diameter, cross section width, or length that is, for example, in the range between 1 nm and 10 nm, in the range between 10 nm and 50 nm, in the range between 50 nm and 100 nm, in the range between 100 nm and 1 μm, in the range between 1 μm and 100 μm, in the range between 100 μm and 1 mm, or in the range between 1 mm and 10 mm. The shell may have a diameter, cross section width, or length that is, for example, between about 1 nm and 10 mm, for example. In some embodiments, the length, width and height of the shell 105 can be approximately equal to each other (e.g., when the shell is in the shape of a sphere), while in others they are not (e.g., when the shell is in the shape of an ellipse). In some embodiments, the shell 105 can be longer than it is wide or tall. For example, the shell 105 may have be at least about, about, or less than about 1.1, 1.2, 1.3, 1.5, 1.8, 2, 2.5, 3, 5, 10, 15 or 20 times longer than it is wide and/or tall. Walls of the shell may be, for example, greater than about, about or less than about 0.1 nm, 0.5 nm, 1 nm, 5 nm, 10 nm, 50 nm, 100 nm, 500 nm, 1 μm, 5 μm thick.

In some embodiments, the shell 105 may only partially surround one or more photocatalytic nanoparticles 110 (e.g., by forming an enclosure with openings or gaps). In some embodiments, partially surround can mean that openings or gaps in the shell 105 may include about 0.001%-1%, 1%-5%, 5%-10%, 10%-20% or 20-50% of the surface area of the shell. In other embodiments, the shell 105 can fully surround the one or more photocatalytic nanoparticles with no opening or gaps. The term “nanoparticle,” as used herein, refers to a particle in which one, more than one, or all dimensions are less than about 1000, 500, 300, 100, 50, 30, 10, 5, 3, or 1 nm in length. In some instances, all dimensions of the nanoparticles can be less than about 1000, 500, 300, 100, 50, 30, 10, 5, 3, or 1 nm in length. The nanoparticles may include, for example, nanospheres, nanorods, nanofibers, nanocubes, etc.

The photocatalytic nanoparticles 110 may be configured to at least partially degrade a contaminant. In some instances, the photocatalytic nanoparticles 110 can at least partially be composed of a photocatalyst or include a photocatalytic material. The photocatalytic nanoparticles 110 can include a water-soluble material and/or an organic material, and/or the photocatalytic nanoparticles 110 can be water soluble and/or organic. The photocatalytic nanoparticles can include a metal oxide. The photocatalytic nanoparticles 110 can include one or more of TiO₂, ZnO, CdS, SrTiO₃, Fe₂O₃, V₂O₅, SnO₂, FeTiO₃, PbO, combinations of the same, and the like. In some embodiments, the photocatalytic nanoparticles can include titanium dioxide (TiO₂) nanoparticles. A material of the photocatalytic nanoparticle may be doped, for example, to make the photocatalytic nanoparticle responsive to a certain spectrum of light energy. For example, TiO₂, which is normally responsive to ultraviolet (UV) light, can be made responsive to visible light by a proper doping. The photocatalytic nanoparticles can include a coating, such as but not limited to a surfactant (e.g., a surface-modifying) coating. Examples of such surfactants and other coatings include aliphatic (COOH-containing) acids. The coating of the photocatalytic nanoparticles prior to the formation of the shell causes the shell to be larger than what it would have been without the coating. The larger shell, in turn, allows the photocatalytic nanoparticles (after the coating has been removed) to move freely inside the shell and also increases the surface area of contact between the nanoparticles and contaminants that enter the shell.

In some instances, such as that shown in FIG. 1A, the photocatalytic nanoparticles 110 are not attached, linked or secured to the shell 105. FIG. 1B shows an example of an embodiment in which at least one of the photocatalytic nanoparticles 110 are attached, linked or secured to (e.g., an inner surface of) the shell 105 for example via an attractive force between silica and the nanoparticles.

In some embodiments, the photocatalytic nanoparticles 110 can be included within a photocatalytic nanoparticle layer 120, as shown in FIG. 1C. The photocatalytic nanoparticle layer may be composed essentially entirely of nanoparticles or it may include additional components and/or materials. The photocatalytic nanoparticle layer 120 may be attached to, linked to, attracted to, bound to and/or positioned on at least part of the shell 105 (e.g., on at least part of an inner surface of the shell). The photocatalytic nanoparticle layer 120 may include a shape similar to (e.g., curved) or different from (e.g., flat) that of the shell 105.

The shell 105 may at least partially surround at least one cavity 115, pore or space, which may include—for example—air or water. The cavity 115 may have a fixed or variable shape. For example, if the photocatalytic nanoparticles 110 are attached to the shell 105 (such as in FIGS. 1B and 1C), the cavity 115 may have a fixed shape, defined e.g. by borders of the photocatalytic nanoparticles 110 (and—in some embodiments—of the shell 105). In another example, if the photocatalytic nanoparticles 110 are not attached to the shell 105 (such as in FIG. 1A), the shape of the cavity 115 may change as the photocatalytic nanoparticles 110 move within the shell.

As shown in FIG. 1D, in some instances, a contaminant 125 may enter the nanocapsule 100 (e.g., through a permeable shell 105). The contaminant 125 may include, for example, inorganic or organic matter. The contaminant may include but is not limited to one or more of acetaldehyde, formaldehyde, toluene, propanal, butene, acetaldehyde, and the like, for example.

Whether or not the contaminant 125 enters the nanocapsule 100 may depend on factors such as whether the contaminant 125 is smaller than the cavity (e.g., in total volume or in certain dimensions), whether the shell 105 is permeable to one or more materials within the contaminant 125 and/or whether the contaminant 125 is smaller (e.g., in at least one dimension) than pores on the shell 105. Thus, one or more dimensions of the nanocapsule 100 and/or permeability characteristics of the shell 105 may be chosen such that selective molecules can enter the nanocapsule 100. In some embodiments, one or more dimensions of the nanocapsule can be chosen based on a predicted or known size of a contaminant 125. For example, the width of the nanocapsule may be large enough to allow entry of the contaminant but small enough such that photocatalytic nanoparticles 110 attached to opposite sides of the shell can contact the contaminant.

In instances in which the positions of one or more photocatalytic nanoparticles 110 are not fixed with respect to the shell 105, the contaminant 125 may displace, e.g., rearrange the positions of, the photocatalytic nanoparticles 110 within the shell upon entry of the nanocapsule 100 to make a room for the contaminant. The contaminant 125 may also displace a fluid (e.g., air or water) previously occupying a cavity 115.

The contaminant 125 may contact or be in close proximity to one or more photocatalytic nanoparticles upon entering the nanocapsule 100. The contact or the close proximity between the contaminant and the photocatalytic nanoparticles is important so that contaminant is in an environment of a high concentration of radicals (e.g., OH—) produced by the photocatalytic process. When the photocatalytic nanoparticles 110 are illuminated with light, photons may be absorbed by the photocatalytic nanoparticles 110, promoting an electron from the valence band to the conduction band, thus producing a hole in the valence band and adding an electron in the conduction band. Although not intending to be limited by a particular theory, the promoted electron may react with oxygen, and a hole remaining in the valence band may react with water, forming reactive radicals, for example, hydroxyl radicals. Thus, radicals produced by the light interacting with the photocatalytic nanoparticles 110 may oxidize the contaminant to water, carbon dioxide, and/or other substances. The photocatalytic particles can also include any other photocatalytic particles that act by a different mechanism or function.

In some embodiments, it can be desirable to have the photocatalytic nanoparticles 110 at least partially mobile with respect to the shell 105 (e.g., as shown in FIG. 1A as compared to embodiments shown in FIGS. 1B-C). In these instances, the photocatalytic nanoparticles may be configured to freely move within the shell, that is to say, it can move a distance that is comparable or larger than its characteristic dimension without being impeded or stopped by other photocatalytic nanoparticles, for example. Thus, when a contaminant 125 is in the nanocapsule, the photocatalytic nanoparticles may contact or come in a close proximity to the contaminant 125 during its traversal, for example through or past a capsule (or fiber(s)). If the photocatalytic nanoparticles degrade the contaminant 125 such that the size of the contaminant 125 begins to decrease, the photocatalytic nanoparticles can continue to contact with or come in a close proximity to the contaminant 125 during its traversal through the shell. Therefore, relative freedom of movement of the photocatalytic nanoparticles 110 as compared to fixed nanoparticles, may increase the number of photocatalytic nanoparticles 110 able to contact the contaminant 125 (and/or the number of photocatalytic contacts), particularly as the size of the contaminant decreases.

Additionally, nanocapsules 100 may be able to effectively degrade contaminants 125 of various sizes especially in the event that the photocatalytic nanoparticles 110 are configured to be at least partially mobile. If the photocatalytic nanoparticles can move, they will likely contact various surfaces especially of e.g. smaller contaminants.

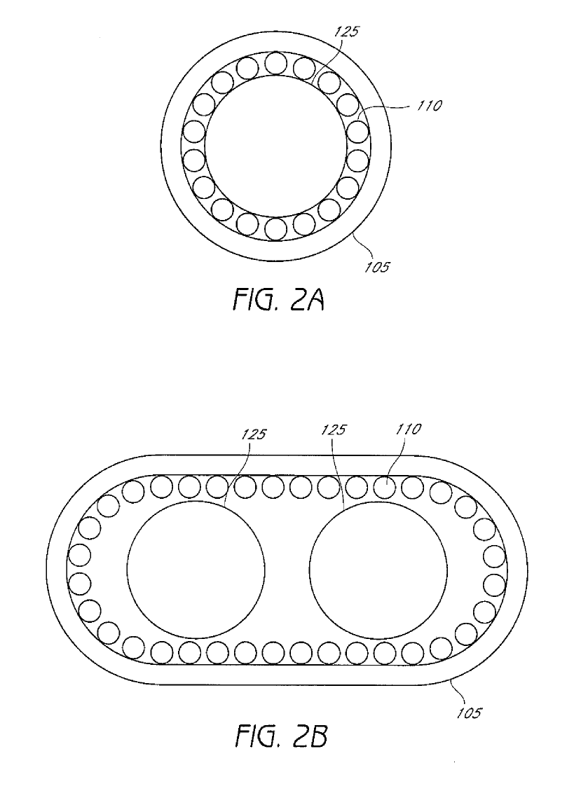

In some embodiments, the size of the nanocapsule 100 can be configured based on a number of contaminant units (e.g., molecules) to be degraded by one nanocapsule and/or size of the contaminant units. FIG. 2A shows an example in which one contaminant 125 can fit inside the nanocapsule. FIG. 2B shows an example where two contaminants 125 can fit inside the nanocapsule. FIGS. 2A and 2B are for illustration only and should not be considered limiting, particularly with respect to the number of contaminants, the number of nanoparticles, the relative sizes or spacing, or the locations of the materials within the capsules. It should be noted that in some instances the nanocapsules can accommodate more than two contaminants, including many more. The number that can be accommodated can depend on the size of the nanocapsule and/or also on the size of the contaminant.

One or more nanocapsules 100 may be used, for example, to clean or sterilize a substance. For example, one or more nanocapsules 100 may contact a substance (e.g., water, air, biological waste, organic waste, etc.). The substance may contain or include contaminants 125. Contaminants 125 of the substance may enter the nanocapsules 100 and contact the photocatalytic nanoparticles 110, which may at least partially decompose or degrade the contaminants.

To promote a photocatalytic activity, light (e.g., ultraviolet or visible light) may be applied to the nanocapsules (e.g., when the substance is contacting the one or more nanocapsules 100) by exposing the photocatalytic nanocapsule to the natural (sun) light or to an artificial light such as from a UV lamp.

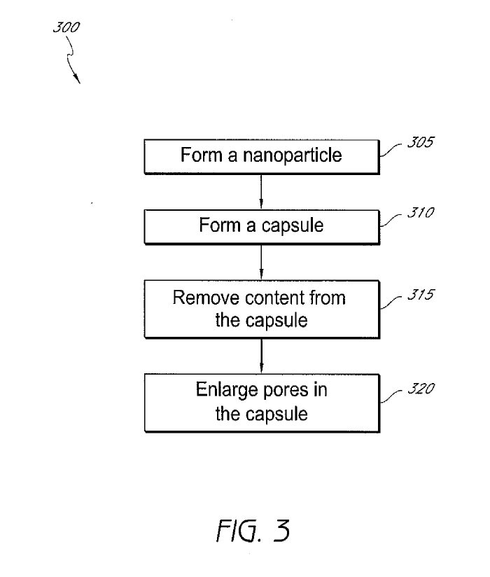

FIG. 3 shows an illustrative process 300 of making a photocatalytic nanocapsule that includes one or more photocatalytic nanoparticles. At step 305, one or more photocatalytic nanoparticles are formed or provided. The photocatalytic nanoparticles may include a photocatalytic nanoparticle described herein, such as but not limited to a titanium or titanium dioxide nanoparticle. In some embodiments, forming the photocatalytic nanoparticles can include hydrolyzing alkoxide in solution. Chloride in aqueous solution is another example. TiO₂ nanoparticles can be formed as the mixture is dehydrated. In certain embodiments, the photocatalytic nanoparticles can be formed in the presence of or in conjunction with a metal oxide (e.g., a metal oxide not contained within the nanoparticle) or a metal sulfide. One method of forming such heterogeneous photocatalytic nanoparticle system includes putting TiO₂ nanorods and metal oxide nanoparticles put into a reaction container or chamber with water and heating the mixture to a temperature of about 100 degrees C. for 24 hours, for example. Ends of certain nanorods, e.g., TiO₂ nanorods, are known to attract other nanomaterials. The attractive force provides a mechanism for anchoring or attaching the metal oxide nanoparticles to the distal ends of the TiO₂ nanorods to form the heterogeneous photocatalytic nanoparticle system. In those embodiments employing the heterogeneous photocatalytic nanoparticle system, the metal oxide or metal sulfide can harvest visible spectrum of sunlight or an artificial light to enhance the photocatalytic activity (PCA) (and hence the contaminant-degradation effectiveness) of the photocatalytic nanocapsules.

At step 310, one or more nanocapsules are formed. The nanocapsules may be formed, for example, using an emulsion technique. In some embodiments, forming a nanocapsule can include forming a shell (e.g., a silicon dioxide shell). Formation of shells around nanoparticles is known in the art. For example, forming of silica-coated iron oxide nanoparticles is described in technical notes of Journal of Proteome Research (PR800067X) by Palani et al., which is incorporated herein by reference in its entirety. The nanocapsules may be configured such that the shell at least partially surrounds one or more of the photocatalytic nanoparticles formed at step 305.

At step 315, content (e.g., surfactant or other coating material) is optionally removed from the nanocapsule. The removal can be achieved by the use of an acid to bring about a chemical or substitution reaction or the use of a base to neutralize an acid-based coating. Alternatively, the content may be removed, for example, by purging or flushing the nanocapsule with a liquid (e.g., water or other solvent) or air. In some embodiments employing polycarbosilane polymers, the removal can be achieved by heating. In some embodiments, the content is content within a cavity of the nanocapsule. The step may selectively remove some types of content (e.g., based on size of the content) or may non-selectively remove all content in the cavity by the use of certain removal techniques that removes certain types of content but not others. In some instances, organic content can be removed.

At step 320, pores of the nanocapsule can optionally be enlarged. The pores may be enlarged using, for example, an etching technique. The etching may be performed in a basic buffer solution. An example method for enlargement of pores is described in the technical notes of Journal of Proteome Research (PR800067X) by Palani et al.

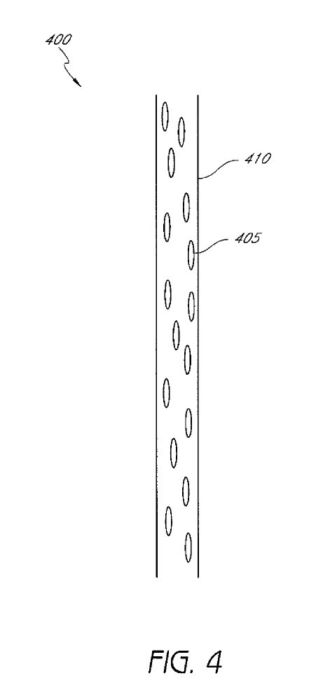

FIG. 4 shows an illustrative photocatalytic fiber 400 that includes one or more embedded photocatalytic nanoparticles 405. In certain embodiments, the photocatalytic fiber can include, but is not limited to, polycarbosilane fibers in which photocatalytic nanoparticles 405, e.g., TiO2 nanoparticles, are trapped or embedded optionally within one or more cavities formed in the polycarbosilane material. In other embodiments, the photocatalytic nanoparticle(s) may be attached to the outside of the polycarbosilane fibers by an attractive force between Si particles of the fibers and the nanoparticles instead of being trapped within the fibers. The nanoparticles can be of any size or shape. For example the fiber can include, but is not limited to, nanorods, nanospheres, nanoellipsoids, nanocubes, combinations of different sizes or shapes, and the like.

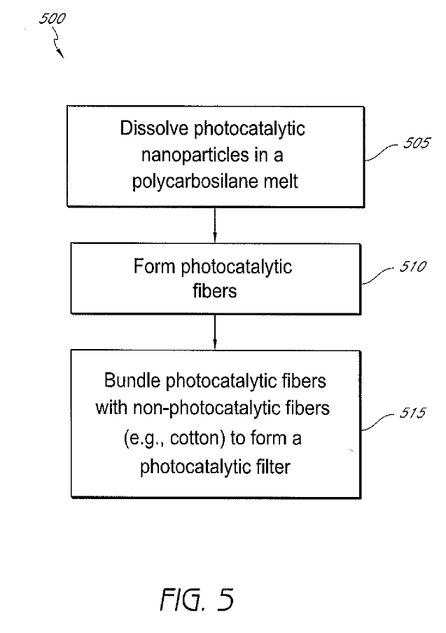

FIG. 5 shows an illustrative process 500 of making a photocatalytic fiber that includes one or more embedded photocatalytic nanoparticles. For example, the photocatalytic fiber 400 shown in FIG. 4 can be made using the example process 500. At step 505, photocatalytic nanoparticles, such as for example TiO₂ nanoparticles, are dispersed in a polycarbosilane melt. Preparation of a polycarbosilane melt suitable for this process is described in detail in Azojomo (Journal of Materials Online) DOI: 10.2240/azojomo0139 (posted September 2005, which is incorporated herein by reference in its entirety. The polycarbosilane can be synthesized from polydimethysilane in the presence of zeolite as a catalyst. Polymers other than the polycarbosilane can be used. In certain embodiments, the photocatalytic nanoparticles can be coated with a surfactant. Photocatalytic fibers including silica or silicon carbide (SiC) fibers can be formed from in the polycarbosilane melt with the photocatalytic nanoparticles dispersed therein by a melt spinning technique. Synthesis of SiC fibers by the melt-spinning of polycarbosilane (PCS), curing, and pyrolysis is described in Composites Science and Technology 59 (1999) 787-792, which is also incorporated herein by reference in its entirety. The melt spinning technique can include electrospinning, for example. Fabrication of nanofibers by electrospinning is described in detail in NANO LETTERS, 2004 Vol. 4, No. 5 933-938, which is also incorporated herein by reference in its entirety. The formation of photocatalytic fibers can include a thermal treatment of polymers (e.g., polycarbosilane) with the dispersed photocatalytic nanoparticles. The resulting photocatalytic fibers can contain holes or cavities made of silica in which the photocatalytic nanoparticles are trapped or embedded. At step 515, the photocatalytic fibers so formed optionally can be bundled (e.g., interwoven intertwined, tied, bonded, fused together, embedded, coated, etc.) to non-photocatalytic fibers such as but not limited to cotton to form a photocatalytic delivery system such as a water or air filter, for example. The relative amount of photocatalytic fiber to non-photocatalytic fiber can be between about 25% to about 99% photocatalytic fiber to 75% to about 1% non-photocatalytic fiber, for example. In some embodiments, the photocatalytic delivery system delivered above can be a water filtration filter. In other embodiments, the system can be used as part of a medical mask or clothing.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A photocatalytic nanocapsule comprising:
one or more photocatalytic nanoparticles;
a shell at least partially composed of silicon dioxide, the shell at least partially encapsulating said one or more photocatalytic nanoparticles and
wherein the shell includes at least one nanopore.

2. The photocatalytic nanocapsule of claim 1, wherein the one or more photocatalytic nanoparticles include one or more titanium dioxide nanoparticles.

3. The photocatalytic nanocapsule of claim 1, wherein the one or more photocatalytic nanoparticles include one or more of ZnO, CdS, SrTiO₃, Fe₂O₃, V₂O₅, SnO₂, FeTiO₃, or PbO.

4. The photocatalytic nanocapsule of claim 1, wherein the one or more photocatalytic nanoparticles are one or more doped photocatalytic nanoparticles.

5. The photocatalytic nanocapsule of claim 1, wherein the shell is configured to allow organic matter to enter through the shell through the at least one nanopore.

6. The photocatalytic nanocapsule of claim 1, wherein the shell is hydrophilic.

7. The photocatalytic nanocapsule of claim 1, comprising two or more photocatalytic nanoparticles at least partially encapsulated by the shell.

8. A photocatalyst comprising two or more of the photocatalytic nanocapsules of claim 1.

9. A method of sterilizing or cleaning comprising:
contacting a substance, the substance including organic matter, with a plurality of photocatalytic nanocapsules of claim 1,
wherein the photocatalytic nanocapsules decompose the organic matter that contacts with or comes in a close proximity to the one or more photocatalytic nanoparticles.

10. The method of claim 9, wherein the substance includes water.

11. The method of claim 9, further comprising: exposing the plurality of photocatalytic nanocapsules to light.

12. The method of claim 11, wherein the light includes visible light.

13. A photocatalytic fiber comprising:
one or more photocatalytic nanoparticles; and
a fiber comprising silica, the silica at least partially surrounding the one or more photocatalytic nanoparticles.

14. The fiber of claim 13, wherein the one or more photocatalytic nanoparticles include one or more nanorods.

15. The fiber of claim 13, wherein the nanoparticles include titanium dioxide.

16. The fiber of claim 13, wherein the nanoparticles are organic.

17. The fiber of claim 13, wherein the nanoparticles are water soluble.

18. The fiber of claim 13, wherein the one or more photocatalytic nanoparticles include a surfactant, wherein the surfactant at least partially coats the nanoparticles.

19. The fiber of claim 13, wherein the fiber is a nanofiber.

20. A plurality of fibers comprising:
at least one photocatalytic fiber of claim 13; and
at least one non-photocatalytic fiber.

21. The plurality of fibers of claim 20, wherein the at least one non-photocatalytic fiber includes cotton.

22. The plurality of fibers of claim 20, wherein the at least one photocatalytic fiber includes a titanium dioxide nanoparticle.

23. A filter comprising the plurality of fibers of claim 20.

24. A method of filtering comprising:
contacting a filter comprising one or more photocatalytic fibers comprising one or more photocatalytic nanoparticles with fluid for filtration, wherein the fluid traverses the filter and contacts the one or more photocatalytic nanoparticles, and
wherein one or more contaminant particles in the fluid are oxidized by one or more photocatalytic nanoparticles.

25. The method of claim 24, wherein the fluid includes water.

26. The method of claim 24, wherein the fluid includes air.

27. A method of making a photocatalytic nanocapsule, the method comprising:
forming a photocatalytic nanocapsule using an emulsion technique, the photocatalytic nanocapsule having one or more photocatalytic nanoparticles at least partially surrounded by a silicon dioxide shell.

28. The method of claim 27, further comprising purging the photocatalytic nanocapsule with air to remove organic content.

29. The method of claim 27, further comprising etching in a basic buffer solution to enlarge nanopores of the photocatalytic nanocapsules.

30. The method of claim 27, further comprising forming the photocatalytic nanoparticles and wherein the forming the nanoparticles includes hydrolyzing alkoxide in solution.

31. The method of claim 30, wherein the photocatalytic nanoparticles are formed in the presence of a non-photocatalytic metal oxide or metal sulfide.

32. The method of claim 27, wherein the photocatalytic nanoparticles include one or more titanium oxide nanoparticles.

33. The method of claim 27, further comprising:
forming a plurality of photocatalytic nanocapsules using an emulsion technique, each photocatalytic nanocapsule comprising one or more photocatalytic nanoparticles and a silicon dioxide shell surrounding the photocatalytic nanoparticles.

34. A method of forming a photocatalytic fiber, the method comprising:
dispersing one or more photocatalytic nanoparticles in a polycarbosilane melt; and
forming at least one photocatalytic fiber from a solution comprising photocatalytic nanoparticles dispersed in the polycarbosilane melt.

35. The method of claim 34, wherein forming the photocatalytic fiber from the solution includes a melt spinning process.

36. The method of claim 34, wherein the fiber is a nanofiber.

37. The method of claim 34, wherein the nanoparticle is a nanorod.

38. The method of claim 34, wherein the photocatalytic nanoparticle is at least partially composed of titanium dioxide nanoparticles.

39. The method of claim 34, wherein the photocatalytic nanoparticle is organic.

40. The method of claim 34, wherein the photocatalytic nanoparticle is water soluble.

41. The method of claim 34, wherein the nanoparticle includes a surfactant coating.

42. A method of forming a photocatalytic filter comprising:
dispersing one or more photocatalytic nanoparticles in a polycarbosilane melt;
forming one or more photocatalytic fibers from a solution comprising the one or more photocatalytic nanoparticles dispersed in the polycarbosilane melt; and
bundling the one or more photocatalytic fibers with one or more non-photocatalytic fibers.

43. The method of claim 42, wherein the one or more photocatalytic nanoparticles include one or more titanium dioxide nanoparticles.

44. The method of claim 42, wherein the one or more non-photocatalytic fibers include cotton.

45. The method of claim 42, wherein the bundling includes interweaving or intertwining the fibers.
